(19) United States
(12) Patent Application Publication
(10) Pub. No.: US 2017/0095146 A1
(43) Pub. Date: Apr. 6, 2017

(54) Combined optical thickness and physical thickness measurement

(71) Applicants: Joshua Noel Hogan, Los Altos, CA (US); Sergey Alexandrov, Galway (IE)

(72) Inventors: Joshua Noel Hogan, Los Altos, CA (US); Sergey Alexandrov, Galway (IE)

(21) Appl. No.: 15/283,347

(22) Filed: Oct. 1, 2016

Related U.S. Application Data
(60) Provisional application No. 62/236,062, filed on Oct. 1, 2015.

(51) Int. Cl. A61B 3/10; A61B 3/103

(52) U.S. Cl. CPC A61B 3/102; A61B 3/1005; A61B 3/103

(57) ABSTRACT

The invention provides a system and method obtaining the refractive index of the cornea. According to the invention, an optical coherence tomography system with a high numerical aperture lens. In a first position, radation is focused on the front surface of the cornea; in a second position, focused on the back surface of the cornea. The distance the high numerical aperture lens moves corresponds to the thickness of the cornea. The OCT reference mirror signals in a first position correspond to the front surface of the cornea; in a second position correspond to the back surface of the cornea. The distance between the first and second positions of the reference mirror corresponds to the optical distance of the cornea. Optical distance divided by physical distance provides the refractive index. Various alternate embodiments are taught.

### CROSS REFERENCES TO RELATED PATENTS OR APPLICATIONS

This application, docket CI150617US, claims priority from provisional patent application No. 62/236062, docket CI150617PR; and is related to U.S. Pat. No. 7,526,329 titled Multiple reference non-invasive analysis system and U.S. Pat. No. 7,751,862 titled Frequency resolved imaging system, the contents of each of which are incorporated by reference herein as if fully set forth herein.

It is also related to U.S. Pat. Nos. 8,870,376 entitled Non Invasive Optical Monitoring and 8,888,284 entitled A Field of Light Based Device, the contents of which are incorporated by reference as if fully set forth herein.

### FIELD OF USE

The invention relates to non-invasive imaging and analysis techniques such as Optical Coherence Tomography (OCT). In particular it relates using optical techniques to monitor or measure attributes of targets such as a human eye and human tissue.

### BACKGROUND OF THE INVENTION

Non-invasive imaging and analysis of targets is a valuable technique for acquiring information about systems or targets without undesirable side effects, such as damaging the target or system being analyzed. In the case of analyzing living entities, such as human tissue, undesirable side effects of invasive analysis include the risk of infection along with pain and discomfort associated with the invasive process. In the case of quality control, it enables non-destructive imaging and analysis on a routine basis.

Optical coherence tomography (OCT) is a technology for non-invasive imaging and analysis. There are more than one OCT techniques. Time Domain OCT (TD-OCT) typically uses a broadband optical source with a short coherence length, such as a super-luminescent diode (SLD), to probe and analyze or image a target. Multiple Reference OCT (MRO) is a version of TD-OCT that uses multiple reference signals. Another OCT technique is Fourier Domain OCT (FD-OCT).

A version of Fourier Domain OCT, called Swept Source OCT (SS-OCT), typically uses a narrow band laser optical source whose frequency (or wavelength) is swept (or varied) over a broad wavelength range. In TD-OCT systems the bandwidth of the broadband optical source determines the depth resolution. In SS-OCT systems the wavelength range over which the optical source is swept determines the depth resolution.

Another version of Fourier Domain OCT, often referred to as Spectral Domain OCT (SD-OCT), typically uses a broad band optical source and a spectrometer to separate out wavelengths and detect signals at different wavelengths by means of a multi-segment detector.

OCT depth scans can provide useful sub-surface information including, but not limited to: measurement of thickness of structures of an eye, such as corneal thickness, lens thickness and retinal thickness; measurement of thickness of layers of tissue; sub-surface images of regions of tissue; magnitude of regions of abnormal tissue growth. More generally OCT depth scans can provide useful sub-surface information regarding attributes of tissue.

In the particular case of measurement of thickness of structures of an eye, such as corneal thickness, OCT scanning and signal processing typically provides the optical thickness of the cornea. The optical thickness is the product of the physical thickness of the cornea and the refractive index of the cornea. If the refractive index is not accurately known, then the physical thickness cannot be known accurately. It follows that an accurate measurement of the optical and physical thickness produces an accurate refractive index.

An accurate refractive index is critical, as many measurements of ocular structures and features depend from the value of the refractive index

There are existing approaches to measuring both the refractive index and the optical thickness, such as are described in patent application (WO2012130818) titled “APPARATUS FOR MODELLING OCULAR STRUCTURES.” However, existing approaches typically require expensive equipment, and complex and bulky systems. Moreover, the time required to perform measurements can be unduly long, relative to the motion of the target, or the endurance of the subject, or other factors.

What is needed is a system and method for rapidly determining the optical thickness and the physical thickness of the cornea, so that the refractive index can be calculated. What is also needed is a single-pass approach whereby the optical and physical thickness of a cornea are determined, and the refractive index calculated therefrom.

### BRIEF SUMMARY OF THE INVENTION

The invention described herein meets at least all of the aforementioned unmet needs. The invention provides a system and method for quantitative assessment of the geometrical, i.e. physical—thickness and the refractive index of the cornea.

In the preferred embodiment, the system comprises an optical coherence tomography system including a high numerical aperture (NA) lens, such that in a first position, the radation is focused on the front surface of the cornea, and in a second position, focused on the back surface of the cornea, and where the distance “D1” the high NA lens moves approximately corresponds to the physical thickness of the cornea modified by the refractive index and curvature of the cornea; and where in a first position of the reference mirror the OCT signals correspond to the front surface of the cornea and in a second position of the reference mirror, the OCT signals correspond to the back surface of the cornea, and where the distance “D2” between the first and second position of the reference mirror corresponds to the optical distance of the cornea.

The optical distance equals the physical distance multiplied by the refractive index. The modified physical thickness of the cornea can be expressed as the actual physical thickness by adding a distance equal to ((N−1).T)/N to correct for the extension of the focal length due to the non-zero refractive index, where N is the refractive index and T is the physical thickness of the cornea.

The two equations involving the two known distances D1, D2 can be solved to provide the two quantities, physical thickness and refractive index of the cornea.

A preferred embodiment of a system according to the invention is comprised of an optical coherence tomography system with a positionable lens so that the Raleigh range of the focused optical coherence tomography beam is less than ten percent of corneal thickness. In a first lens position, radation is focused on the front corneal surface; in a second lens position, radiation focused on the back corneal surface. The distance between the first and second lens positions provides a measure of the modified physical thickness of cornea reduced by the refractive index.

The system further includes a positionable reference mirror, such that interference signals received when the reference mirror is in a first mirror position correspond to the front surface of said cornea, and interference signals received when the reference mirror is in a second mirror position correspond to the back surface of said cornea. The distance between the first and second mirror positions provides a measure of the optical distance of said cornea.

Combining the corneal thickness modified by refractive index of the cornea and the optical distance of the cornea provides the refractive index of the cornea and the physical thickness of said cornea.

In an alternate embodiment, the system includes a VFL—variable focal length—lens which is electrically refocusable.

### BRIEF DESCRIPTION OF THE DRAWINGS

The drawings provided as an aid to understanding the invention are:

FIG. 1 depicts collimated beams from an optical coherent tomography system, focused upon the cornea and measuring the optical distance of a cornea.

FIG. 2 depicts an optical coherence tomography system with a high Numerical Aperture (NA) lens focusing a collimated beam on the front surface of the cornea of an eye according to the invention.

FIG. 3 depicts an optical coherence tomography system with a high Numerical Aperture (NA) lens as in FIG. 2 focusing a collimated beam on the back surface of the cornea of the eye, and the distance said lens moves, which distance enables calculation of the physical thickness of cornea according to the invention.

FIG. 4 illustrates a detail of FIG. 3 wherein the distance the high NA lens moves is weakly dependent on the refractive index of the cornea and the curvature of the cornea.

FIG. 5 depicts a variable focal length lens (VFL) in an alternate embodiment of the inventive system.

FIG. 6 depicts employment of a variable focal length lens according to an alternate embodiment of the invention.

### DETAILED DESCRIPTION OF A PREFERRED EMBODIMENT

Terminology used herein is intended to be the commonly understood meaning in the art areas of optical coherence tomography and ophthalmology. As used herein, numerical aperture is abbreviated “NA.” High NA means an NA such the Raleigh range of the focused beam will be significantly smaller (less than 10%) than the thickness of the cornea. Variable focal length—abbreviated as “VFL”—lens means an electronically focusable lens. The term “optical coherence tomography system” is understood by those of average skill in the art. Optical coherence tomography is often abbreviated as “OCT.”

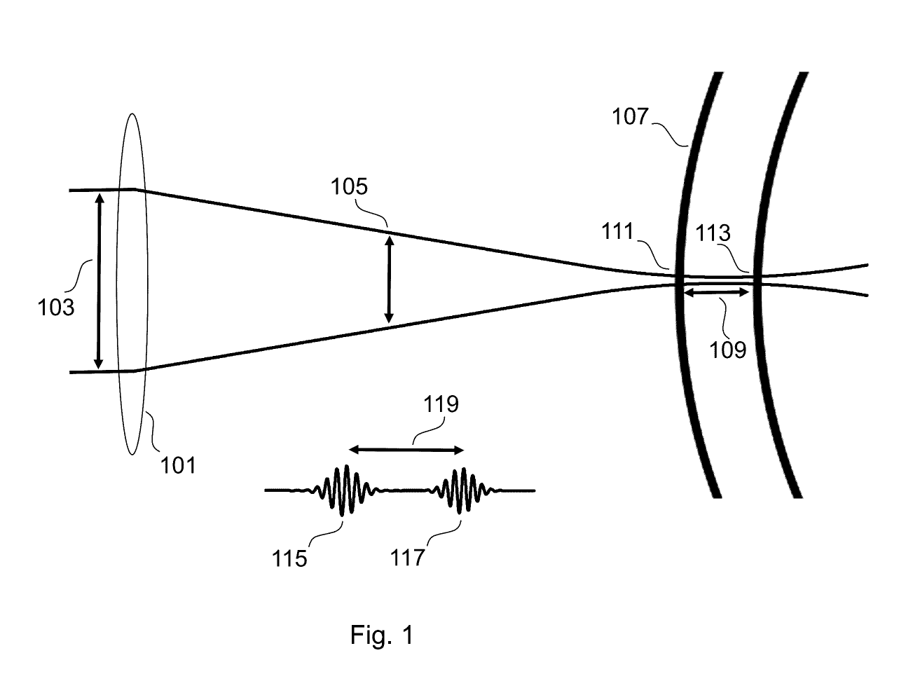

Referring now to FIG. 1, a collimated beam 103 passes through a lens 101 and thereupon the focusing beam 105 aimed on a cornea 107, with a front surface 111 and a back surface 113, with the distance 109 indicative of a focusing with a long Raleigh range. The entire optical coherence tomography system is not shown. The optical signal from the front surface 111 of the cornea 107 is depicted as 115. The optical signal from the back surface 113 of the cornea 107 is depicted as 117, and the distance between the signals, 119, is the optical distance- the optical thickness—of the cornea.

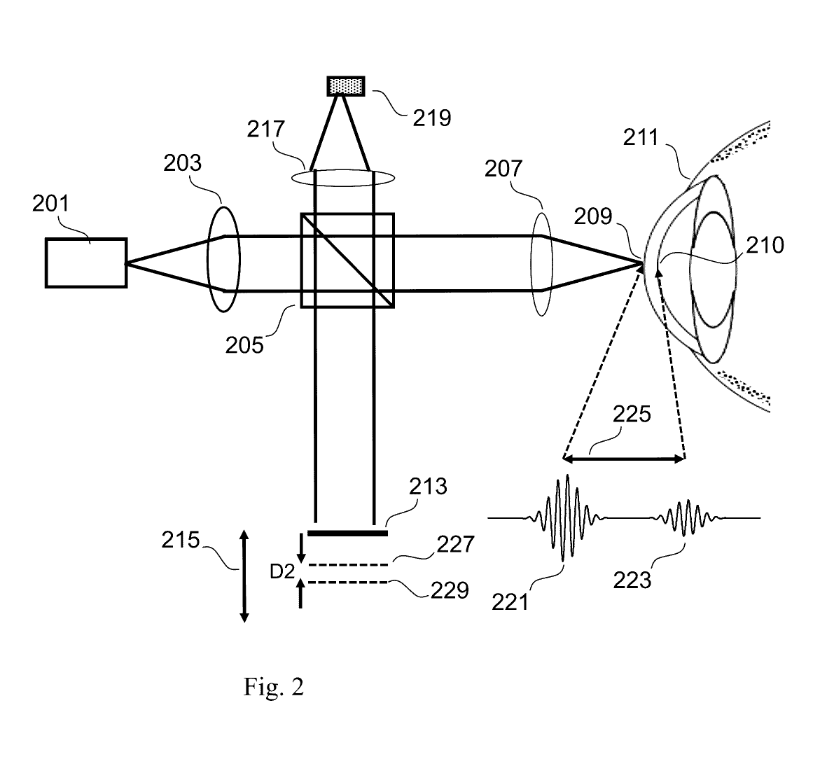

Referring now to FIG. 2, an optical coherent tomography (OCT) system 200 with a high numerical aperture (NA) lens 207 is depicted. The OCT system 200 comprises an optical source 201 which emits a broad band radiation which is collimated by a first lens 203; a first lens 203, a beam splitter 205, a second lens 217 between the beam splitter 205 and the detector 219; a high numerical aperture (NA) lens 207 between the beam splitter and the cornea 211. The configuration illustrates focusing the beam on the front surface 209 of the cornea 21, and the optical distance 225. The reference mirror 213 moves through a distance 215, where mirror position 227 corresponds to the front surface 209 of the cornea, and 221 depicts interference signals from the front surface 209; and mirror position 229 corresponds to the back surface 210 of the cornea 211, and 223 depicts interference signals from the back surface. The signals from the back surface 223 are weaker than the signals from the front surface 221.

The distance D2 between 227 and 229 is equal to the optical thickness 225. FIG. 2 illustrates that the optical thickness can be determined by measuring the distance between peaks 221 and 223. Because the mirror scan path distance 215 is known, the peaks of the interference signals can be measured. Thus the OCT system with a focusing element readily measures the optical thickness of the cornea.

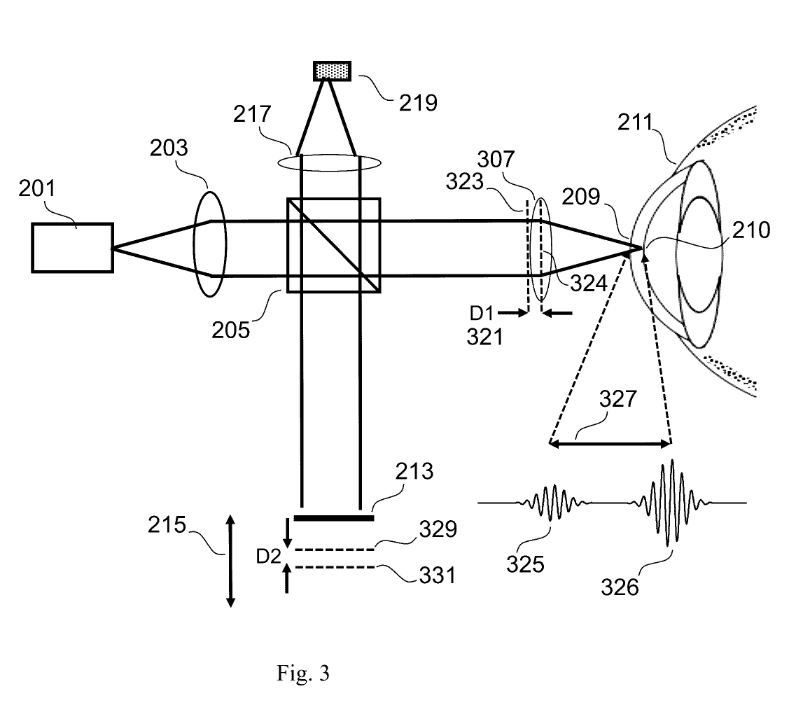

Referring now to FIG. 3, the OCT system of FIG. 2 is depicted where the high numerical aperture (NA) lens 307 moves from a first position 323 where the beam focus is on the front surface 209 of the cornea 211, to a second position 324, where the beam focus is on the back surface 210 of the cornea. The distance D1 between the first position and the second position 321 of the high NA lens provides an approximation of the physical thickness of the cornea. Similar to FIG. 2, the optical distance is 327. The distance 215 is the scan range of the reference mirror. The reference mirror moves through a first position 329 corresponding to the interference signal 325 from the front surface 209 of the cornea, through a second position 331 corresponding to the interference signal 326 from the back surface 210 of the cornea. The signals from the front surface 325 are weaker than the signals from the back surface 326.

Determining the positions from the location of the maximum of the envelope of the interference signals provides the precise location of 329, 331, and 323 and 324. Conventional signal processing provides the point of the maximum of the envelope of 325 and of 323 (for example using a polynomial fit), thus providing an accurate location of 329 and 331.

Continuous scanning moves the lens through a range, and then signal processing of the acquired scan data identifies the maximums. Thus to find out where to locate 323, maximize the magnitude of envelope of 325, and to find 324, maximize the envelope of 326.

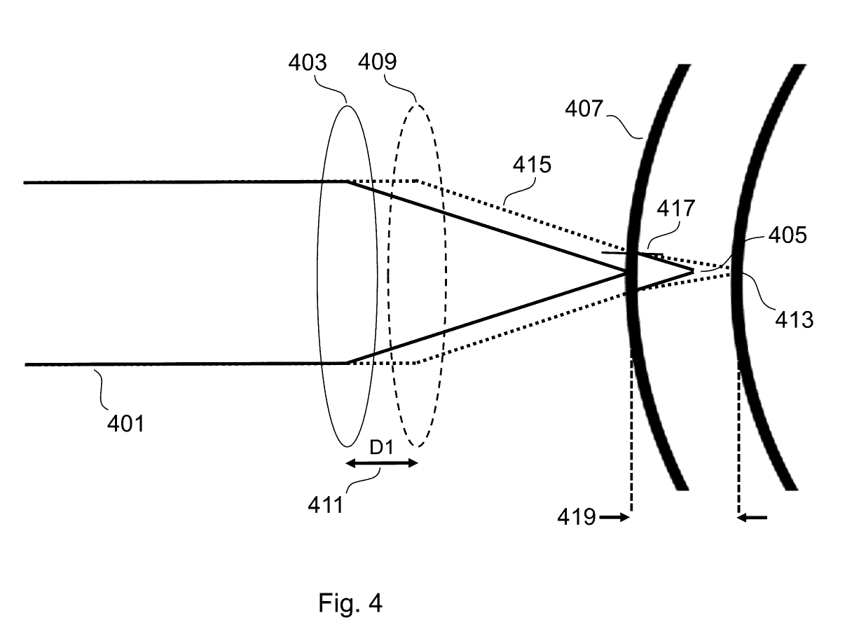

FIG. 4 schematically illustrates the change in beam angle at the front surface of the cornea 407, point 417, as it proceeds to the back surface 413 of the cornea. A first lens position 405 focuses the beam 410 on the front surface of the cornea 407. A second lens position 409 directs the beam through the front surface of the cornea 407 (dotted line 415) and focusing on the back surface of the cornea 413. The point 405 indicates where the focus of the lens in the second position 409 would be if the refractive index of the cornea were zero.

The incident angle at the front corneal surface 417 depicts the diffractive refractive angle of the beam within the cornea. The change in angle demonstrates a dependence of the physical distance the high NA lens moves (see 411 in FIG. 4; 321 in FIG. 3) on the refractive index of the cornea. Consequently the distance D1 the high NA lens moves approximates the physical thickness of the cornea 419 divided by the refractive index of the cornea. The figure is illustrative and the distances not to scale. It can be appreciated that subsequent signal processing can provide any correction that may be needed to compensate for this slight angular change.

The measured value D1 is equal to the corneal physical thickness T divided by the refractive index N of the cornea. D1=T/N.

The measured value D2 is the corneal physical thickness multiplied by the refractive index of the cornea.

D2=T×N.

Given

D1=T/N and D2=T×N

Then

N=Square root of (D2/D1)

The refractive index of the cornea N is equal to the square root of D2 divided by D1. Once N is known, the corneal physical thickness T is readily calculated.

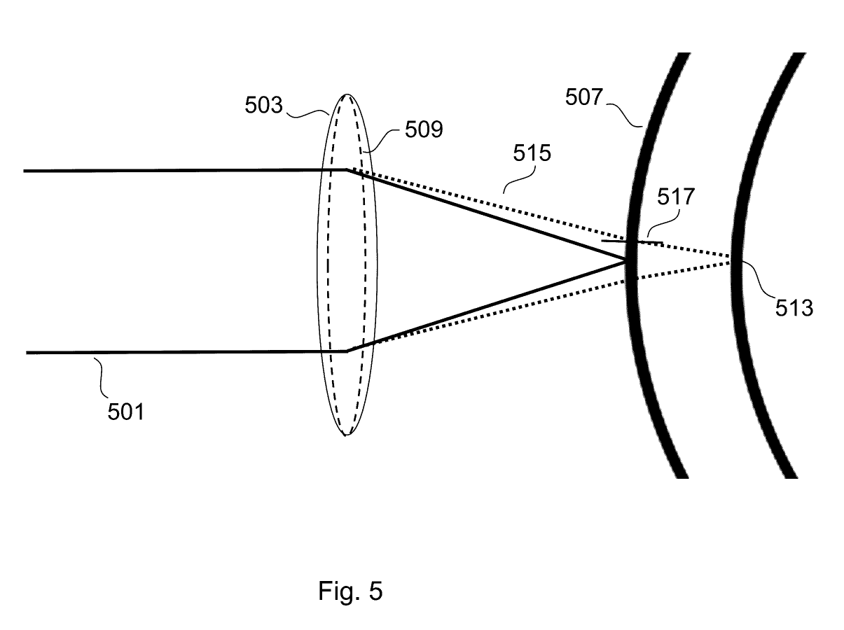

In an alternate embodiment depicted in FIG. 5, the system provides an electrically focusable lens—also referred to as a variable focusable lens, often abbreviated VFL—such that from a single position, the lens is focusable on either the front or back surface of the cornea. Numbered elements are generally the same as in FIG. 4, i.e. 501 is the same as 401. Hence, 503 is a first configuration of lens with a first focal length focused on the front surface of the cornea 407. The second configuration of the lens 509 with a second focal length (beam depicted by 515) focuses on the back surface of the cornea 513. From the two voltages and the calibration curve of the VFL, the equivalent measurement D1 can be found.

As described above once D1 and D2 are known T, the corneal thickness, and N, the refractive index of the cornea, can be calculated.

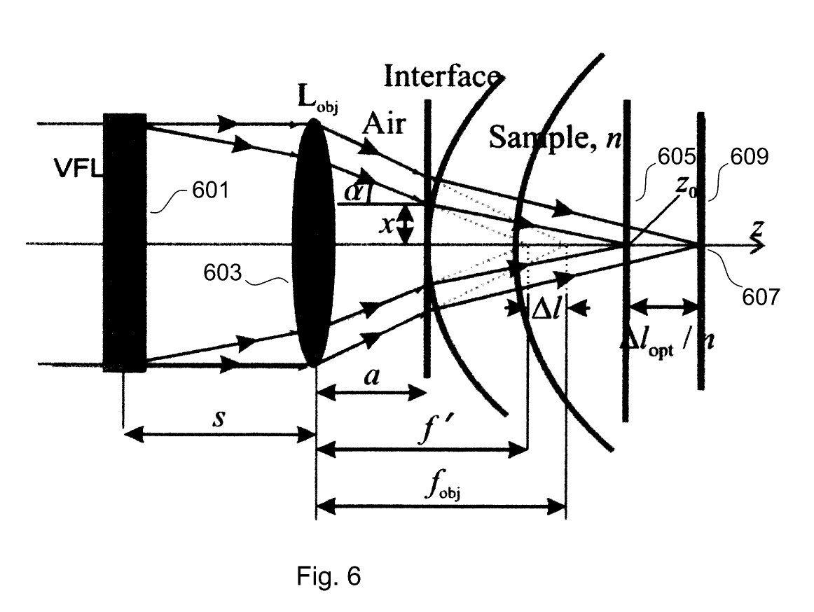

Referring now to FIG. 6, a schematic of the illumination system with VFL with a low NA is depicted in combination with a conventional lens with high NA comprising a compound lens with a sufficiently high combined NA.

In an alternate embodiment, when a VFL is used in conjunction with a time domain OCT system, the frequency with which the VFL is focused on the front and rear surfaces can be synchronized with the frequency of the depth scanning rate of the OCT system and the timing adjusted such that the VFL is focused on the front surface of the cornea when the OCT system is scanning the front surface and the VFL is focused on the rear surface of the cornea when the OCT system is scanning the rear surface.

This synchronized method also can be used to increase the penetration depth of the OCT at high lateral resolution.

In other embodiments the thickness and refractive index of eye components other than the cornea are measured. For example, the thickness and refractive index of the crystalline lens can be measured.

FIG. 6 depicts a VFL 601 with a low NA in conjunction with a conventional high NA lens 603 at a separation “s” and a distance “a” in front of the cornea. FIG. 6 also depicts the light forused at at “z₀” on the front surface 603 of the crystalline lens and also, when the VFL has a different applied voltage, at point 605 on the rear surface 607 of the crystalline lens.

In other embodiments, optical and geometrical distances between any two surfaces can be measured and so calculate the local refractive indices (by reconstructing the refractive index profiles within the scattering sample). For example the surfaces of contact lenses for quality control purposes.

In other embodiments, optical and geometrical distances between any two scatterers within the image can be measured and so calculate the local refractive indices (by reconstructing the refractive index profiles within the scattering sample). Examples include regions of malignant tissue, and contaminants in non-biological specimens.

It can be appreciated by one of skill in the art that variations of the embodiments of the invention taught herein exist, though not set forth herein. The scope of this invention, therefore, should be determined with reference to the specification in its entirety and the drawings, along with the entire range of equivalents as applied thereto.

I claim:

1. A system comprising:
an optical coherence tomography system with a positionable lens, such that the Raleigh range of the focused optical coherence tomography beam is less than ten percent the thickness of a human cornea, and wherein
in a first lens position, radation is focused on the front surface of said cornea;
in a second lens position, radiation is focused on the back surface of said cornea, and where the distance between said first lens position and said second lens position provides a measure of the thickness of said cornea reduced by the refractive index;
a positionable reference mirror, such that interference signals received when said reference mirror is in a first mirror position correspond to the front surface of said cornea;
and interference signals received when said reference mirror is in a second mirror position correspond to the back surface of said cornea, and where the distance between said first mirror position and said second mirror position provides a measure of the optical distance of said cornea;
wherein combining said thickness of said cornea reduced by refractive index and said optical distance provides the refractive index of said cornea and the physical thickness of said cornea.

2. The system of claim 1 wherein said positionable lense is an electrically refocusable lens, and at a first voltage said lens focuses on the front surface of said cornea under test, and at a second voltage said lens focuses on the back surface of said cornea under test.
